(19) United States
(12) Patent Application Publication
(10) Pub. No.: US 2025/0205311 A1
(43) Pub. Date: Jun. 26, 2025

(54) PHARMACEUTICAL COMPOSITIONS OF SEMAGLUTIDE AND THE METHODS OF USE THEREOF

(71) Applicant: PENTIDE THERAPEUTICS LIMITED, Hong Kong (CN)

(72) Inventors: Yanfeng WANG, Hong Kong (CN); Liang XU, Hong Kong (CN); Wai Han LAM, Hong Kong (CN)

(21) Appl. No.: 18/849,043

(22) PCT Filed: Apr. 28, 2023
(86) PCT No.: PCT/IB2023/054435

§ 371 Date: Sep. 20, 2024

Related U.S. Application Data
(60) Provisional application No. 63/337,031, filed on Apr. 29, 2022.

(51) Int. Cl. A61K 38/26; A61K 9/00; A61K 9/08; A61K 47/10; A61K 47/18; A61K 47/24; A61K 47/36; A61K 47/38; A61K 47/69; A61P 3/04; A61P 3/10

(52) U.S. Cl. CPC A61K 38/26; A61K 9/0043; A61K 9/08; A61K 47/10; A61K 47/183; A61K 47/24; A61K 47/36; A61K 47/38; A61K 47/6951; A61P 3/04; A61P 3/10

(57) ABSTRACT

Pharmaceutical compositions of semaglutide or its salts for intranasal administration in the treatment of diabetes mellitus, obesity, nonalcoholic fatty liver disease (NAFLD), or neurodegenerative diseases. A method comprising the delivery of semaglutide via intranasal route avoids repeated injections and improves systemic absorption as compared to an oral tablet.

### FIELD OF THE INVENTION

The present invention relates to pharmaceutical compositions of semaglutide and the salts for intranasal administration, and the use in the treatment of diabetes mellitus, obesity, nonalcoholic fatty liver disease (NAFLD), or neurodegenerative diseases.

### BACKGROUND OF THE INVENTION

Semaglutide (C187H291N45059) is a long-acting GLP-1 receptor agonist with 94% structural similarity to the native GLP-1 (Knudsen et al., 2019), it is also known as N6,26-{18-[N-(17-carboxyheptadecanoyl)-L-glutamyl]-1 0-oxo-3,6, 12, 15-tetraoxa-9, 18-diazaoctadecanoyl}-[8-(2-amino-2-propanoic acid), 34-L-arginine] human glucagon-like peptide 1 (7-37), as described in WO 2020/084126. The preparation method of semaglutide is described in WO2006/097537, Example 4. Semaglutide may be present in the compositions in fully or partly ionized form.

Semaglutide is an anti-diabetic medication used for treatment of type 2 diabetes and chronic weight management. Semaglutide acts like human glucagon-like peptide-1 (GLP-1) and results in the elevation of insulin secretion, thereby increasing sugar metabolism. Semaglutide has been found to reduce hyperglycaemia, body weight, steatosis, and improves cognitive ability in neurodegenerative diseases (Mahapatract al, 2022). Currently two semaglutide products are approved by USFDA, Health Canada, European Medicines Agency, Japanese Health ministry for use in treatment of type 2 diabetes, including Ozempic® (subcutaneous injection, weekly-once dosing; available in 0.5, 1.0 mg strength) and Rybelsus® (oral tablets, once-daily dosing; available in 3, 7, 14 mg strength).

The use of Ozempic® injection pen is complicated and patients need to be trained by experienced medical staffs. Long-term and frequent injection of semaglutide is inconvenient and uncomfortable to patients, associated with risk of infections and injection site reactions (e.g. erythema), see FDA approved package insert of Ozempic®. Semaglutide oral tablet provides alternative treatment option for the patients unwilling or unable to self-inject glucose lowering medicines; however, despite the aforementioned advantages, the oral bioavailability of semaglutide is very low and highly variable (0.4-1%) due to the low permeability and extensive degradation and metabolism in gastrointestinal (GI) tract. To achieve similar systemic absorption, the weekly dose of oral semaglutide tablet is 49 to 98 mg, much higher (approximately 100-fold) than the injection dose of 0.5 to 1.0 mg per week; also the oral tablets have to be taken daily at least 30 minutes before the first food or drink; the high dose of permeation enhancer (SNAC) of 300 mg per tablet may cause gastrointestinal adverse reactions like nausea, abdominal pain and vomiting, see FDA approved package insert of Rybelsus®.

In view of the disadvantages for marketed semaglutide products, a drug delivery system for nasal mucosa is an alternative and promising choice since the drug can directly enter into blood circulation from absorption sites, thus high GI degradation and liver metabolism can be completely bypassed, as well as convenience and acceptance to the general public. The advantages of intranasal semaglutide include but are not limited to: non-invasiveness, convenience and case of use enabling patient self-dosing; ready and complete absorption directly via thin nasal epithelium to blood circulation and resulting in rapid onset of action; bypassing GI degradation and first-pass metabolism with high bioavailability; a reduced dose with less dosing frequency (i.e. 1-2 doses weekly) as compared to oral dosage form; minimal local or systemic adverse effects; no drug-drug interaction and food effect; no dose adjustment required in special populations.

The nasal liquid compositions of liraglutide, a GLP-1 agonist, was disclosed in WO2007/146488, to maintain the physical and chemical stability of the formulations, the pH was adjusted to 8.5 or higher, which is much higher than the physiological pH of nasal mucus (5.5-6.5), and will cause local irritating effects for chronic use. In addition, liraglutide is a short-acting GLP-1 agonist, with subcutaneous dose of 1.25-1.9 mg per day, based on the pharmacokinetics results in rabbit, the inventors concluded that the drug concentration up to 50 mg/mL have to be used, with 2-4 doses per day, the frequent nasal dosing will potentially result in toxic effects, as well as poor patient compliance.

WO2007/0611434 described pharmaceutical compositions of intranasal exenatide using similar formulation technologies in WO2007/146488, plus a dipeptidyl aminopeptidase (DPP) IV inhibitor to minimize enzymatic degradation of exenatide. Given the short half-life of exenatide (2.4 hours), the effective drug concentration in blood circulation, as well as the glucose lowing effect, can be only maintained for 3-4 hours, indicating at least 6-8 intranasal doses daily, therefore, such composition is not suitable to be used clinically for treating metabolic syndrome in mammal.

### SUMMARY OF THE INVENTION

The present invention relates to pharmaceutical compositions of semaglutide and its salts for intranasal administration in the treatment of diabetes mellitus, obesity, nonalcoholic fatty liver disease (NAFLD), or neurodegenerative diseases by intranasal administration. The delivery of semaglutide via intranasal route avoids repeated injections, and improves systemic absorption as compared to marketed oral tablet.

### BRIEF DESCRIPTION OF THE DRAWING

FIG. 1. Mean semaglutide plasma concentration versus time profiles after intranasal administration of PT-N01, PT-N02, PT-N03 at single dose of 0.2 mg/animal in rats.

FIG. 2. Mean semaglutide plasma concentration versus time profiles after intranasal administration of PT-N01, PT-N04, PT-N05 at single dose of 4 mg/animal and subcutaneous administration of PT-S01 at a single dose of 0.25 mg/animal in rabbits.

FIG. 3. Mean blood glucose versus time profiles after intranasal administration of PT-N01, PT-N04, PT-N05 at single dose of 4 mg/animal and subcutaneous administration of PT-S01 at a single dose of 0.25 mg/animal in rabbits.

FIG. 4. Mean body weight versus time profiles after intranasal administration of PT-N01, PT-N04, PT-N05 at single dose of 4 mg/animal and subcutaneous administration of PT-S01 at a single dose of 0.25 mg/animal in rabbits.

FIG. 5. Mean food intake versus time profiles after intranasal administration of PT-N01, PT-N04, PT-N05 at single dose of 4 mg/animal and subcutaneous administration of PT-S01 at a single dose of 0.25 mg/animal in rabbits.

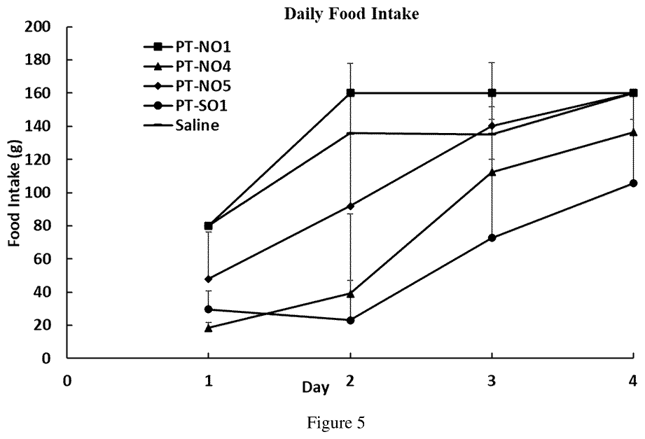

FIG. 6. Mean semaglutide plasma concentration versus time profiles after intranasal administration of PT-N06 at single dose of 7 mg/animal and an oral dose of Rybelsus® Tablet 7 mg in beagle dogs.

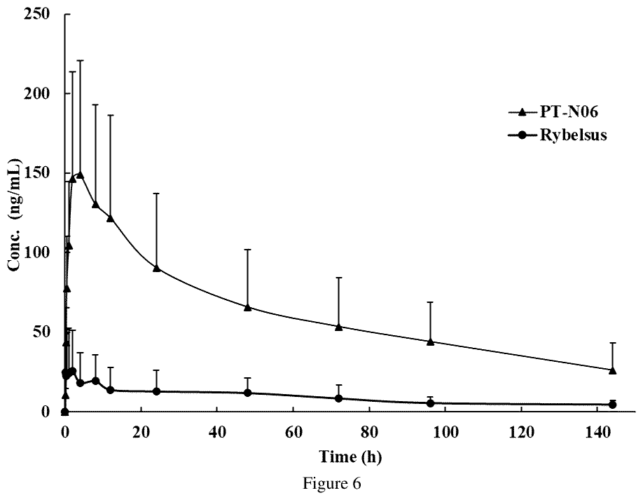

### DETAILED DESCRIPTION OF THE INVENTION

The invention relates to the pharmaceutical compositions capable of delivering sufficient dose of semaglutide via intranasal routes. The marketed oral semaglutide results in couples of undesirable effects, such as high first-pass effect with very low bioavailability, high inter-subject variation, drug-drug interactions and food effect, GI adverse reactions caused by permeation enhancer. The present intranasal compositions enable semaglutide to be quickly absorbed through nasal mucosa, thereby the GI degradation and metabolism can be completely bypassed, thus the above disadvantages via the oral administration can be well addressed with rapid and improved absorption, case of use and minimal side effects.

The composition according to the present invention includes the active ingredient, i.e., semaglutide or a pharmaceutically salt thereof. Semaglutide used in current invention includes both in the form of free base or the pharmaceutically acceptable salts. The pharmaceutically acceptable salts include, but not limited to sodium, potassium, calcium, magnesium, lithium, cesium, palladium, ammonium. The preferable salt used in this invention is semaglutide sodium, which is formed between semaglutide and sodium with a mass ratio from 1:1 to 100:1.

The use of semaglutide or a pharmaceutically salt according to the present invention includes formulations wherein the treatment dosage of semaglutide is delivered to the nasal mucosa. The preferred formulations are the liquid dosage forms, including a solution, suspension, emulsion, bioadhesive or in-situ gel, microsphere, nanoparticle, self-emulsifying drug delivery system; or the solid dosage forms, including powders, granules; or the semi-solid dosage forms, including ointments, creams, hydrogel; or other forms suitable for intranasal delivery in the art.

Semaglutide is a hydrophilic compound with relatively high molecular mass (MW: 4113.64), exceeding the cutting-off molecular weight for intranasal drug delivery (Pathak K., 2011), therefore it is expected that the nasal absorption of semaglutide is quite poor. In our pharmacokinetics and pharmacodynamics study in rabbits, the intranasal bioavailability of semaglutide aqueous solution is around 0.09%, and drug blood concentration is far below the therapeutic level even though the high dose of 4 mg per animal was administered intranasally; therefore, it is imperative to improve intranasal absorption utilising various mucosal drug delivery technologies.

Surprisingly, the present invention shows that the bioavailability of semaglutide can be greatly improved by comprehensively utilising multiple permeation enhancing agents, including but not limited to: i) Cell penetrating peptides (CPPs) as vector to enable transcellular drug transport; ii) Tight junction modulating agents to increase paracellular permeation; iii) Bioadhesive polymeric agents to prolong the mucosal residence time and minimise nasal cilia clearance to the drug.

One important aspect of the present invention is to facilitate semaglutide transcellular transport by aid of Cell penetrating peptides (CPPs). It was found that the intranasal absorption of semaglutide can be remarkably improved by incorporation of cell perpetrating peptides (CPPs) by physical mix or covalent conjugation with semaglutide molecule. CPPs are positively charged short peptides with 5-30 amino acids long that can penetrate into biological membrane and used as a novel carrier for intracellular delivery (Derakhshankhah H, Jafari S. 2018). CPPs have received extensively investigated deliver macromolecules due to their high intracellular transduction efficiency and also low cytotoxicity. The preferred CPPS in current invention include but not limited to TAT, R6, R8, Penetratin, Protamine, Transportan, and their derivatives. CPPS can form complex with semaglutide and significantly enhance the delivery across nasal epithelium cells through endocytosis process.

Another important aspect of the present invention is to further improve paracellular transport of semaglutide across nasal mucosa by tight junction modulating agents. The tight junction modulating agents in present invention can be selected from: (i) Phospholipid surfactants, including dodecylphosphocholine (DPC), 1,2-didecyl phosphatidylcholine (DDPC), 1,2-distearoyl-sn-glycero-3-phosphocholine (DSPC), 1-didecanoy 1-sn-glycero-3-phosphocholine (LLPC), 1,2-dioctanoyl-sn-glycero-3-phosphocholine (D8PC), 1-1-palmitoyl-2-glutaroyl-sn-glycero-3-phosphocholine (PGPC), the preferred phospholipid surfactants are DPC and DSPC, due to the better solubility and stability in liquid formulations, as well as low mucosal toxicity; (ii) Cyclodextrin derivatives, including alpha-cyclodextrins, beta-cyclodextrin, di-methyl-beta-cyclodextrin, hydroxypropyl-beta-cyclodextrin, gamma-cyclodextrin, preferred cyclodextrin derivatives are di-methyl-beta-cyclodextrin and hydroxypropyl-beta-cyclodextrin; (iii) Chelators, including EDTA, EGTA, BAPTA, preferred chelator is EDTA; (iv) Bile salts, including sodium cholate, dehydrocholate, taurocholate; (v) fatty acids and phosphate esters, including oleic acid, sodium caprate, palmitoyl carnitine, lysophosphatidic acid; (vi) Cationic polymers, such as chitosan derivatives and protamine; (vii) Surfactants, including sodium dodecyl sulphate (SDS), Tween 20; and (viii) Nitric oxide donors, or bradykinin.

A major hurdle in nasal delivery is the rapid removal of drug formulations (aqueous solution or dry powder) from the nasal cavity by rapid mucociliary beating, resulting in a clearance half-life of about 15 min for ordinary formulations, as well as low bioavailability (Merkus et al., 1998), especially for semaglutide and other polypeptides with high molecular mass and low diffusion rate across mucosal epithelium. Hence, mucoadhesion, including bioadhesive/mucoadhesive agents and/or in-situ gelling agents should be considered to allow prolonged retention time, extended drug release and sustained therapeutic effect.

In some embodiments of the invention, pharmaceutically acceptable bioadhesive/mucoadhesive agent is selected from a group consisting of methylcellulose, hydroxymethyl cellulose, hydroxypropyl cellulose, hydroxy propylmethyl cellulose, carboxymethyl cellulose, hyaluronic acid, sodium alginate, chitosan, gelatin, lectin, poly(acrylic acid), acacia, carbopol 934P, xanthan gum, guar gum, and carrageenan, and the combinations thereof.

The in-situ gelling agents are water soluble polymers with bioadhesive properties and capable of changing the rheological behaviour in relation to ion, pH and temperature, and can form non-Newtonian fluid that is free flowing in spray device, when being mixed or sprayed, then forms a thick gel. In some embodiments of the invention, pharmaceutically acceptable in-situ gelling agent in is selected from a group consisting of poloxamer, gellan gum, pectin. carbomer, carrageenan, cellulose acetate phthalate, and the combinations thereof.

The first aspect relates to pharmaceutical compositions comprising semaglutide in a suitable formulation at a dose of 0.01 mg to 100 mg. The composition is suitable for intranasal administration, typically to administer intranasally by aid of a nasal spray device.

Pharmaceutically acceptable buffering agents may be used to maintain the optimal pH conditions for achieving physicochemical stability and minimizing local irritation to nasal mucosa. The suitable pH range according to the present invention ranges from 3.0 to 9.0, preferably 4.0 to 7.0. The preferred buffering systems include without limitation to phosphate buffer, acetic buffer, boric buffer, citrate buffer, tartaric buffer, and tris buffer.

The present compositions also contain one of the pharmaceutical preservatives include but not limit to: benzalkonium chloride, benzethonium chloride, benzyl alcohol, chlorobutanol, chlorhexidine, methylparaben and propylparaben, phenylethyl alcohol, phenylmercuric acetate, thimerosal. The preferred preservatives without adverse effect on nasal mucosa include but not limited to benzyl alcohol, benzalconium chloride, chlorhexidine, and thimerosal.

Finally, the compositions of the present invention may also contain: (1) chelators, i.e. sodium EDTA; (2) antioxidant, i.e. sodium metabisulphite; (3) tonicity agents, including dextrose, glycerine, hydroxypropyl betadex, mannitol, sorbitol, potassium chloride, and sodium chloride.

The semaglutide or a pharmaceutically salt thereof compositions, preferably in aqueous or semi-solid form, are sprayed into nasal cavity using a non-pressurized disperser. Suitable dispenser includes a spray pump and a bottle, and can deliver a single dose or multiple doses by mechanical actuation. A spray volume ranges from 10 to 200 μL, more preferably from 50 to 150 μL, and most preferably from 80 to 120 μL in each nostril.

A further aspect of the invention relates to use of a treatment dosage of semaglutide comprising 0.1 to 50 mg semaglutide in a suitable pharmaceutical vehicle for intranasal delivery, for preparation of a medicament for treatment of diabetes mellitus, obesity, nonalcoholic fatty liver disease (NAFLD), or neurodegenerative diseases.

Both pharmacokinetics and pharmacodynamics profiles after intranasal administration of semaglutide pharmaceutical compositions (PT-N01 to PT-N06) in rats, rabbits and beagle dogs are described in EXAMPLEs 3 to 5. According to the inventors' knowledge, there is no patent or publication showing the delivery of semaglutide via intranasal route, with the unexpected rapid and significantly improved adsorption over the marketed oral tablet (Rybelsus®). Based on the animal pharmacokinetic results, the intranasal dose can be further reduced from oral dose of 7-14 mg daily to intranasal dose of 8-10 mg weekly or less, but systemic absorption and efficacy will be comparable or superior to oral tablets, with the faster control of blood glucose and body weight, and is free of GI toxicity, food effect and drug-drug interactions.

### EXAMPLES

### Example 1. Semaglutide Nasal Sprays

PT-N01. 200 mg semaglutide was completely dissolved in 10 mL PBS 7.0 buffer solution by stirring at ambient temperature. Then the solution was filtered through a 0.45 μm filter membrane and filled into a glass bottle fitted with a metered-dose spray pump for intranasal application in a volume of 0.10 mL/spay. 2 mg semaglutide will be delivered intranasally per spray.
PT-N02. In one exemplary composition of the invention with ingredients listed in Table 1, a nasal spray composition was prepared using one of cell penetration peptides-penetratin as the permeation enhancer.

TABLE 1
Ingredients of PT-N02

| Ingredients | Amount | Unit |
|---|---|---|
| Semaglutide | 60 mg | mg |
| Penetratin | 60 mg | mg |
| PBS 5.0 buffer solution | q.s. to 6 mL | mL |

### Preparation Process:

(a) Charge 60 semaglutide into a glass vial equipped with a magnetic stir bar.
(b) Add an appropriate amount of PBS 5.0 buffer solution and dissolve the API by stirring at room temperature.
(c) Add 60 mg Penetratin into the solution and stir the mixture till completely dissolved.
(d) Check and adjust the solution pH to 5.0 with HCl or NaOH solution.
(e) Add PBS 5.0 buffer solution to the required volume (5 mL).
(f) Filter the solution through a 0.45-micron filter.
(g) Pour the solution into a spray nasal dispenser with a volume of 3.5 nil and the applicator delivers a quantity comprising 1.2 mg semaglutide per actuation (0.1 mL).
PT-N03. In one exemplary composition of the invention, a nasal spray composition with ingredients listed in Table 2 was prepared using the permeation enhancers like n-dodecylphosphocholine (DPC), 2,6-dimethyl-β-cyclodextrin and edetate disodium (EDTA-2Na).

TABLE 2
Ingredients of PT-N03

| Ingredients | Amount | Unit |
|---|---|---|
| Semaglutide | 100 | mg |
| n-Dodecylphosphocholine | 300 | mg |
| 2,6-dimethyl-β-Cyclodextrin | 1000 | mg |
| Disodium Edetate | 100 | mg |
| Hydroxypropyl methylcellulose | 50 | mg |
| PBS 6.5 buffer solution | q.s. to 10 | mL |

### Preparation Process:

(a) Charge semaglutide into a glass vial equipped with a magnetic stir bar.
(b) Add an appropriate amount of PBS 6.5 buffer solution and dissolve the API by stirring at room temperature.
(c) Add n-Dodecylphosphocholine into the solution and stir the mixture till completely dissolved.
(d) Add 2,6-dimethyl-β-Cyclodextrin and disodium edetate into the vial and mix the solution till dissolved.
(e) Add HPMC into the vial and dissolve the excipient by stirring.
(f) Add PBS 6.5 buffer solution to the required volume (10 mL).
(g) Check and adjust the solution pH to 6.5 with HCl or NaOH solution.
(h) Filter the solution through a 0.45-micron filter.
(i) Pour the solution into a spray nasal dispenser with a volume of 3.5 mL and the applicator delivers a quantity comprising 1 mg semaglutide per actuation (0.1 mL).
PT-N04. In one exemplary composition of the invention, a nasal spray composition with ingredients listed in Table 3 was prepared using the permeation enhancers like n-dodecylphosphocholine (DPC), 2,6-dimethyl-β-cyclodextrin and edetate disodium (EDTA-2Na). The bioadhesive polymer like hydroxypropyl methylcellulose (HPMC K100LV) is applied to prolong the residence time of the drug in nasal cavity. Firstly, 200 mg semaglutide was completely dissolved in about 8 mL PBS 7.0 buffer solution at ambient temperature. Then DPC, 2,6-dimethyl-β-cyclodextrin and EDTA.2Na were added and dissolved in the buffer solution by stirring at ambient temperature. After that, HPMC was added and stirred till dissolved. Finally, q.s. PBS 7.0 buffer solution was added to 10 mL. The concentration of semaglutide in the final solution is 20 mg/mL.

TABLE 3
Ingredients of PT-N04

| Ingredients | Amount | Unit |
|---|---|---|
| Semaglutide | 200 | mg |
| DPC | 300 | mg |
| 2,6-dimethyl-β-cyclodextrin | 1000 | mg |
| EDTA-2Na | 100 | mg |
| Hydroxypropyl methylcellulose | 30 | mg |

| | |
|---|---|
| PBS 7.0 buffer (50 mM) | Q.S. to 10 mL |

Following preparation, the drug solution was filtered through a 0.22 μm filter membrane and was then filled into a glass bottle fitted with a metered-dose spray pump for intranasal application in a volume of 0.10 mL/spay. In which 2 mg semaglutide will be delivered intranasally per spray.
PT-N05. Semaglutide and the absorption enhancer, N-(8-[2-hydroxybenzamido]) sodium caprylate (SNAC), were completely dissolved in about 8 mL PBS 7.0 buffer solution at ambient temperature. Then dimethyl-β-cyclodextrin and edetate disodium (EDTA-2Na) were added into the solution as permeation enhancers. Hydroxypropyl methylcellulose (HPMC K100LV) was added into the solution and stirred till completely dissolved at ambient temperature. The final solution with ingredients listed in Table 4 was made to 10 mL with q.s. PBS 7.0 buffer solution. The concentration of semaglutide is 20 mg/mL in final spray solution.

TABLE 4
Ingredients of PT-N05

| Ingredients | Amount | Unit |
|---|---|---|
| Semaglutide | 200 | mg |
| SNAC | 300 | mg |
| 2,6-dimethyl-β-cyclodextrin | 1000 | mg |
| EDTA-2Na | 100 | mg |
| Hydroxypropyl methylcellulose | 30 | mg |

| | |
|---|---|
| PBS 7.0 buffer (50 mM) | Q.S. to 10 mL |

Following preparation, the drug solution was filtered through a 0.22 μm filter membrane and was then filled into a glass bottle fitted with a metered-dose spray pump for intranasal application in a volume of 0.10 mL/spay. In which 2 mg semaglutide will be delivered intranasally per spray.
PT-N06. Semaglutide and the absorption enhancer, n-dodecylphosphocholine (DPC), were completely dissolved in about 8 mL PBS 7.0 buffer solution at ambient temperature. Edetate disodium (EDTA-2Na) were added into the solution. Hydroxypropyl methylcellulose (HPMC K100LV) was added into the solution and stirred till completely dissolved at ambient temperature. The final solution with ingredients listed in Table 5 was made to 10 mL with q.s. PBS 7.0 buffer solution. The concentration of semaglutide is 35 mg/mL in final spray solution.

TABLE 5
Ingredients of PT-N06

| Ingredients | Amount | Unit |
|---|---|---|
| Semaglutide | 350 | mg |
| DPC | 300 | mg |
| EDTA-2Na | 100 | mg |
| Hydroxypropyl methylcellulose | 30 | mg |

| | |
|---|---|
| PBS 7.0 buffer (50 mM) | Q.S. to 10 mL |

Following preparation, the drug solution was filtered through a 0.22 μm filter membrane and was then filled into a glass bottle fitted with a metered-dose spray pump for intranasal application in a volume of 0.10 mL/spay. In which 3.5 mg semaglutide will be delivered intranasally per spray.

### Example 2. Additional Semaglutide Nasal Sprays

PT-B01. In one composition of the invention with ingredients listed in Table 6, a bioadhesive nasal spray was prepared using carboxymethylcellulose sodium (CMC-Na) as a thickener.

TABLE 6
Ingredients of PT-B01

| Ingredients | Amount (for 100 mL) | |
|---|---|---|
| Semaglutide | 2 | g |
| n-Dodecylphosphocholine | 3 | g |
| Disodium Edetate | 1 | g |
| CMC-Na | 0.5 | g |

| | |
|---|---|
| PBS 6.5 buffer solution | q.s. to 100 mL |

### Preparation Process:

(a) Charge semaglutide into a glass vial equipped with a magnetic stir bar.
(b) Add phosphate buffer solution (pH 6.5) into the glass bottle and dissolve the API by stirring at room temperature.
(c) Add n-Dodecylphosphocholine into the solution and stir the mixture till dissolved at room temperature.
(d) Add Disodium Edetate and CMC-Na and dissolve the excipients by stirring at room temperature.
(e) Add PBS 6.5 buffer solution to the required volume (100 mL).
(f) Check and adjust the solution pH to 6.0-7.0 by HCl or NaOH solution.
(g) Filter the solution through a 0.45-micron filter.
(h) Pour the solution into a spray nasal dispenser with a volume of 3.5 mL and the applicator delivers a quantity comprising 2 mg semaglutide per actuation (0.1 mL).

### Semaglutide Nasal Spray Containing Sodium Hyaluronate.

PT-B02. In one composition of the invention with ingredients listed in Table 7, a bioadhesive nasal spray was prepared using sodium hyaluronate.

TABLE 7
Ingredients of PT-B02

| Ingredients | Amount (for 100 mL) | |
|---|---|---|
| Semaglutide | 1 | g |
| n-Dodecylphosphocholine | 3 | g |
| Disodium Edetate | 1 | g |
| Sodium hyaluronate | 0.2 | g |

| | |
|---|---|
| PBS 6.5 buffer solution | q.s. to 100 mL |

### Preparation Process:

(a) Charge n-Dodecylphosphocholine into a glass vial equipped with a magnetic stir bar.
(b) Add an appropriate amount of PBS 6.5 buffer solution and stir the mixture till dissolved at room temperature.
(c) Add semaglutide into the solution and stir the mixture till completely dissolved at room temperature.
(d) Add disodium edetate into the solution and dissolve the excipient by stirring at room temperature.
(e) Add sodium hyaluronate into the solution and stir the mixture solution till completely dissolved at room temperature.
(f) Add PBS 6.5 buffer solution to the required volume (100 mL).
(g) Check and adjust the solution pH to 6.0-7.0 by HCl or NaOH solution.
(h) Filter the solution through a 0.45-micron filter.
(i) Pour the solution into a spray nasal dispenser with a volume of 3.5 mL and the applicator delivers a quantity comprising 1 mg semaglutide per actuation (0.1 mL).

### Semaglutide Nasal Spray Containing a Poloxamer.

PT-B03. In one composition of the invention with ingredients listed in Table 8, a nasal spray composition was prepared using a poloxamer.

TABLE 8
Ingredients of PT-B03

| Ingredients | Amount (for 100 mL) | |
|---|---|---|
| Semaglutide | 2 | g |
| n-Dodecylphosphocholine | 3 | g |
| Disodium Edetate | 1 | g |
| Poloxamer 188 | 1 | g |
| Poloxamer 407 | 18 | g |

| | |
|---|---|
| PBS 6.5 buffer solution | q.s. to 100 mL |

### Preparation Process:

(a) Charge semaglutide into a glass vial equipped with a magnetic stir bar.
(b) Add an appropriate amount of PBS 6.5 buffer solution into the vial and dissolve the API by stirring at room temperature.
(c) Add n-Dodecylphosphocholine into the solution and stir the mixture till completely dissolved at room temperature.
(d) Add disodium edetate into the solution and dissolve it by stirring at room temperature.
(e) Add Poloxamer 188 into the solution and stir in an ice bath till dissolved completely. Then add Poloxamer 407 into the solution and dissolve it by stirring in an ice bath.
(f) Add PBS 6.5 buffer solution to the required volume (100 mL).
(g) Check and adjust the solution pH to 6.0-7.0 by HCl or NaOH solution.
(h) Place the solution at 4° C. overnight until obtain a clear solution.
(i) Filter the solution through a 0.45-micron filter.
(j) Pour the solution into a spray nasal dispenser with a volume of 3.5 mL and the applicator delivers a quantity comprising 2 mg semaglutide per actuation (0.1 mL).

### Bioadhesive Semaglutide Nasal Spray Containing Carbomer 934P.

PT-B04. In one composition of the invention with ingredients listed in Table 9, a nasal spray composition was prepared using carbomer 934P.

TABLE 9
Ingredients of PT-B04

| Ingredients | Amount (for 100 mL) | |
|---|---|---|
| Semaglutide | 2 | g |
| n-Dodecylphosphocholine | 3 | g |
| Disodium Edetate | 1 | g |
| Carbomer 934P | 0.2 | g |

| | |
|---|---|
| PBS 6.5 buffer solution | q.s. to 100 mL |

### Preparation Process:

(a) Charge semaglutide into a glass vial equipped with a magnetic stir bar.
(b) Add an appropriate amount of PBS 6.5 buffer solution into the vial and dissolve the API by stirring at room temperature.
(c) Add n-Dodecylphosphocholine into the solution and stir the mixture till completely dissolved.
(d) Add disodium edetate into the solution and stir the mixture till dissolved.
(e) Add carbomer 934P into the above solution and stir the mixture till dissolved completely.
(f) Add PBS 6.5 buffer solution to the required volume (100 mL).
(g) Check and adjust the solution pH to 6.0-7.0 by HCl or NaOH solution.
(h) Filter the solution through a 0.45-micron filter.
(i) Pour the solution into a spray nasal dispenser with a volume of 3.5 mL and the applicator delivers a quantity comprising 2 mg semaglutide per actuation (0.1 mL).

### Bioadhesive Semaglutide Nasal Spray Containing Chitosan Hydrochloride.

PT-B05. In one composition of the invention with ingredients listed in Table 10, a nasal spray composition was prepared using Chitosan Hydrochloride (Chitosan. HCl).

TABLE 10
Ingredients of PT-B05

| Ingredients | Amount (for 100 mL) | |
|---|---|---|
| Semaglutide | 3.5 | g |
| n-Dodecylphosphocholine | 3 | g |
| Disodium Edetate | 1 | g |
| Chitosan•HCl | 0.3 | g |

| | |
|---|---|
| PBS 6.5 buffer solution | q.s. to 100 mL |

### Preparation Process:

(a) Charge semaglutide into a glass vial equipped with a magnetic stir bar.
(b) Add an appropriate amount of PBS 6.5 buffer solution into the vial and dissolve the API by mixing at room temperature.
(c) Add n-Dodecylphosphocholine and disodium edetate into the solution and stir the mixture till completely dissolved.
(d) Add Chitosan HCl into the solution and dissolve the excipient by stirring at room temperature.
(e) Add PBS 6.5 buffer solution to the required volume (100 mL).
(f) Check and adjust the solution pH to 6.0-7.0 by HCl or NaOH solution.
(g) Pass the solution through a 0.45-micron filter.
(h) Pour the solution into a spray nasal dispenser with a volume of 3.5 mL and the applicator delivers a quantity comprising 3.5 mg semaglutide per actuation (0.1 mL).

### Bioadhesive Semaglutide Nasal Spray Containing Hydroxypropyl Cellulose.

PT-B06. In one composition of the invention with ingredients listed in Table 11 a nasal spray composition was prepared using Hydroxypropyl Cellulose (HPC).

TABLE 11
Ingredients of PT-B06

| Ingredients | Amount (for 100 mL) | |
|---|---|---|
| Semaglutide | 3.5 | g |
| n-Dodecylphosphocholine | 3 | g |
| Disodium Edetate | 1 | g |
| Hydroxypropyl Cellulose (HPC) | 0.5 | g |

| | |
|---|---|
| PBS 6.5 buffer solution | q.s. to 100 mL |

### Preparation Process:

(a) Charge Semaglutide and n-Dodecylphosphocholine in a glass vial equipped with a magnetic stir bar.
(b) Add an appropriate amount of PBS 6.5 buffer solution and mix the mixture till completely dissolved at room temperature.
(c) Add disodium edetate into the solution and dissolve it by mixing.
(d) Add HPC into the solution and dissolve the excipient by mixing at room temperature.
(e) Add phosphate buffer solution (pH 6.5) to the required volume (100 mL).
(f) Check and adjust the solution pH to 6.0-7.0 by HCl or NaOH solution.
(g) Pass the solution through a 0.45-micron filter.
(h) Pour the solution into a spray nasal dispenser with a volume of 3.5 nil and the applicator delivers a quantity comprising 3.5 mg semaglutide per actuation (0.1 mL).

### Semaglutide Subcutaneous Injection.

PT-S01. 20 mg semaglutide was completely dissolved in 20 mL PBS 7.0 buffer solution by stirring at ambient temperature. Then the solution was filtered through a 0.22 μm filter membrane. Finally, the filtrate was filled into a glass bottle for subcutaneous injection. The concentration of semaglutide injection is 1 mg/mL.

### Example 3. Pharmacokinetic Study on Semaglutide Composition in Rats

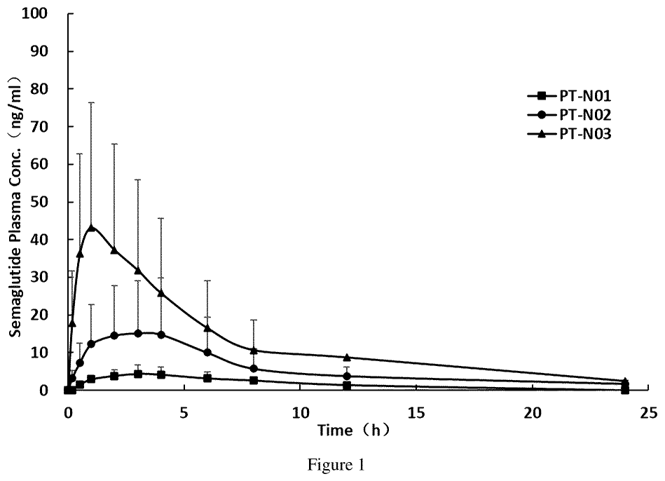

The study in this example is aimed at investigating the intranasal absorption of semaglutide liquid formulations. SD rats were assigned into 3 groups (n=3 per group), each group received an intranasal dose (0.2 mg/rat) of solution composition (PT-N01, PT-N02, or PT-N03), which was prepared according to the Example 1. Multiple blood samples were collected from tail vein until 24 hrs. Semaglutide concentration in rat plasma was determined using a validated LC-MS method. The standard non-compartmental method was used to generate the pharmacokinetic parameters. FIG. 1 shows the mean semaglutide plasma concentration versus time profiles after intranasal administration of PT-N01, PT-N02, PT-N03. Pharmacokinetic parameters are summarized in Table 12. In comparison to the very low intranasal absorption of PT-N01, semaglutide in PBS solution, the incorporation of permeation enhancers, Penetration (PT-N02) or n-Dodecylphosphocholine (DPC), results in the earlier and higher semaglutide concentration in rat plasma, and the bioavailability is also significantly improved by either Penetratin or DPC.

TABLE 12
Pharmacokinetic parameters of semaglutide after intranasal spray of
PT-N01, PT-N02, and PT-N03 and subcutaneous injection of PT-S01.

| | | PT-N01 | | PT-N02 | | PT-N03 | | PT-S01 | |
|---|---|---|---|---|---|---|---|---|---|
| Parameters | Unit | AVG | SD | AVG | SD | AVG | SD | AVG | SD |
| Kₑₗ | h⁻¹ | 0.047 | 0.021 | 0.027 | 0.006 | 0.030 | 0.000 | 0.0200 | 0.000 |
| t_{1/2} | h | 16.4 | 5.9 | 25.0 | 4.5 | 22.2 | 1.6 | 37.1 | 4.1 |
| Tₘₐₓ | h | 6.00 | 5.29 | 3.33 | 1.15 | 2.67 | 2.89 | 32.00 | 13.86 |
| Cₘₐₓ | ng/mL | 18.2 | 6.08 | 429 | 180 | 159 | 108 | 634 | 132 |
| Vz/F | mL | 153748 | 61284 | 9513 | 4748 | 54468 | 68559 | 263 | 65 |
| Cl_F | ml/h | 6458 | 508 | 266 | 118 | 1655 | 2053 | 5 | 1 |
| AUC₀₋ₜ | h*ng/mL | 605 | 58 | 16030 | 6960 | 5919 | 4639 | 44114 | 8231 |
| AUC_{0-inf} | h*ng/mL | 622 | 50 | 17328 | 7926 | 6227 | 4857 | 51797 | 7299 |
| MRT | h | 27.7 | 5.4 | 29.4 | 1.7 | 28.5 | 1.9 | 45.5 | 3.5 |

(N = 3)

### Example 4. Pharmacokinetics and Pharmacodynamics of Semaglutide after Nasal and Subcutaneous Administration in Rabbits

The objective is to study the pharmacokinetics and pharmacodynamics of semaglutide after intranasal spray and subcutaneous injection. Rabbits (n=3 for each dose) received single intranasal dose (4 mg/animal) of three nasal spray compositions (PT-N01, PT-N04, PT-N05) which was prepared according to the Example 1 of this invention, or single subcutaneous injection dose (0.25 mg/animal) of PT-S01 which was prepared according to the Example 2 of this invention. Multiple blood samples were collected from ear vein at 0 (pre-dose), and 0.5, 1, 2, 4, 6, 8, 12, 24, 48, 72, 96, 144 hr post-dose. Semaglutide concentration in rabbit plasma was determined using a validated LC/MS/MS method. The standard non-compartmental method was used to generate the pharmacokinetic parameters. Blood glucose was tested at 0 (pre-dose), 0.5, 1, 2, 4, 6, 8, 12, 24, 48, 72, 96, and 144 hr post-dose, body weight and food intake were also recorded once daily.

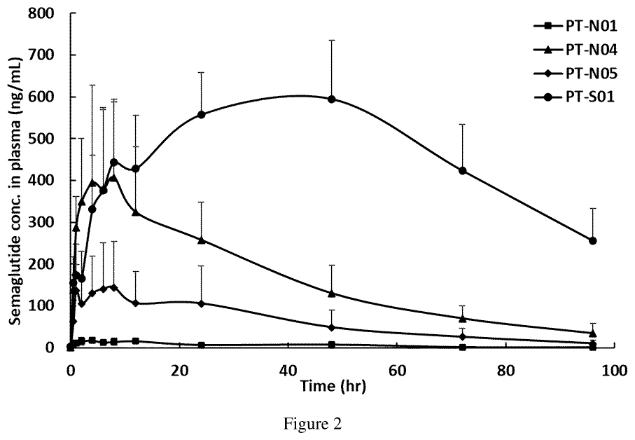

FIG. 2 shows the mean semaglutide plasma concentration versus time profiles after intranasal administration of PT-N01, PT-N04, PT-N05 at single dose of 4 mg/rabbit and subcutaneous administration of PT-S01 at a single dose of 0.25 mg/rabbit. Pharmacokinetic parameters are summarized in Table 13. The results indicate that the intranasal administration of our optimal formulation (PT-N04) can achieve comparable drug plasma concentration to single subcutaneous dose of solution composition, and the bioavailability of PT-N04 is around 28 times of the semaglutide dissolved in PBS (PT-N01). In comparison, less enhancement effects was achieved (around 9 times) using SNAC as permeation enhancer (PT-N05).

TABLE 13
Pharmacokinetic parameters of semaglutide after intranasal instillation
of PT-N01, PT-N02, and PT-N03 at the dose of 0.2 mg/rat.

| | | PT-N01 | | PT-N02 | | PT-N03 | |
|---|---|---|---|---|---|---|---|
| Parameters | Unit | AVG | SD | AVG | SD | AVG | SD |
| Kₑₗ | h⁻¹ | 0.160 | 0.017 | 0.163 | 0.046 | 0.133 | 0.040 |
| t_{1/2} | h | 4.383 | 0.474 | 4.440 | 1.421 | 5.477 | 1.403 |
| Tₘₐₓ | h | 2.667 | 0.577 | 2.667 | 1.528 | 1.000 | 0.000 |
| Cₘₐₓ | ng/mL | 4.44 | 2.22 | 15.8 | 14.1 | 43.2 | 33.1 |
| Vz/F | mL | 43239 | 32165 | 16391 | 12367 | 12482 | 14280 |
| Cl_F | ml/h | 6541 | 4129 | 2989 | 2613 | 2045 | 2733 |
| AUC₀₋ₜ | h*ng/mL | 29.8 | 18.8 | 121 | 126 | 281 | 219 |
| AUC_{0-inf} | h*ng/mL | 38.3 | 18.8 | 132 | 129 | 299 | 228 |
| MRT | h | 4.56 | 1.01 | 4.82 | 1.50 | 4.93 | 1.63 |

(N = 3)

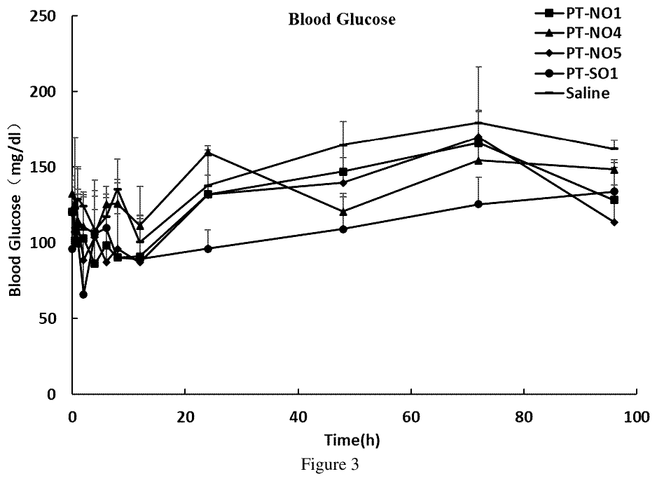

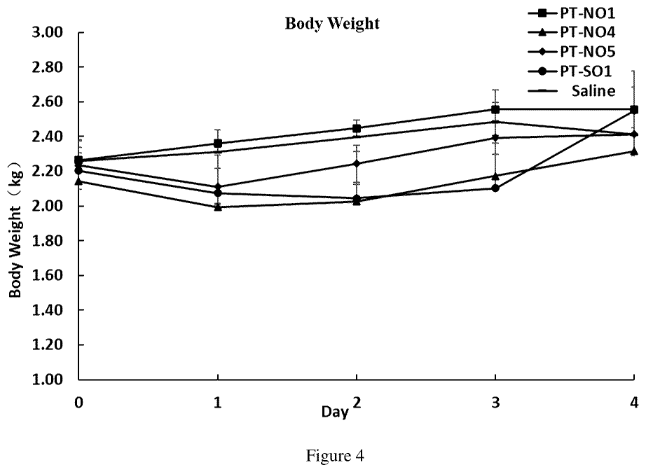

Pharmacological effects including blood glucose, body weight, and food intake are presented in FIG. 2 to FIG. 4. Body weight dropped over 15% on Day 2 after single intranasal dose of PT-N04, which is comparable to that of subcutaneous PT-S01 group; the daily food intake reduced over 70% during Day 2-3 after single intranasal dose of PT-N04, which is also comparable to that of subcutaneous PT-S01 group. In comparison to the fluctuated and elevated blood glucose in saline and PT-N01 groups, more stable blood glucose levels over the study period were observed in PT-N05 and PT-S01 groups.

### Example 5. Pharmacokinetics of Semaglutide after Nasal and Oral Administration in Beagle Dogs

The objective is to compare the pharmacokinetics of semaglutide after single intranasal spray of PT-N06 (7 mg) and single oral dose of Rybelsus® Tab (7 mg). Pharmacokinetic parameters are presented in Table 14. Eight Beagle dogs (4 male and 4 female) participated in the study. Dogs were fasted overnight prior to drug administration, water was given ad libitum throughout the study, Food was provided 4 hours post-dose. Group 1 (2 male and 2 female) received single intranasal dose (7 mg/animal) of nasal spray PT-N06 which was prepared according to the Example 1 of this invention; Group 2 ((2 male and 2 female) received single oral dose of Rybelsus® Tablet (7 mg/animal). Multiple blood samples were collected from ear vein at 0 (pre-dose), 0.167, 0.333, 0.5, 1, 2, 4, 8, 12, 24, 48, 72, 96, and 144 hr post-dose. Semaglutide concentration in dog plasma was determined using a validated LC/MS/MS method. The standard non-compartmental method was used to generate the pharmacokinetic parameters. Blood glucose, body weight and food intake were also recorded during the study period.

TABLE 14
Pharmacokinetic parameters of semaglutide after intranasal spray of PT-
N06, or oral dose of Rybelsus ® Tablet. (N = 3-4)

| | G1 (PT-N06) | | G2 (Rybelsus ®) | |
|---|---|---|---|---|
| PK Parameter | AVE | SD | AVE | SD |
| Tₘₐₓ (h) | 3.33 | 1.15 | 3.63 | 5.59 |
| Cₘₐₓ (ng/mL) | 149.6 | 70.8 | 24.9 | 27.7 |
| AUC₀₋ₜ (h*ng/mL) | 8993 | 4868 | 1170 | 1210 |
| AUC_{0-∞} (h*ng/mL) | 11547 | 6880 | 1950 | 1260 |
| t_{1/2} (h) | 62.8 | 11.7 | 82.7 | 46.5 |
| Vd/F (mL) | 70167 | 41674 | 624000 | 588000 |
| CL/F (mL/h) | 832 | 598 | 4550 | 2280 |
| MRT (h) | 88.67 | 15.39 | 115 | 63.5 |

### REFERENCES

1. Knudsen L B, Lau J. The Discovery and Development of Liraglutide and Semaglutide. Front Endocrinol (Lausanne). 2019; 10:155.
2. Mahapatra M K, Karuppasamy M·Sahoo B M. Therapeutic Potential of Semaglutide, a Newer GLP-1 Receptor Agonist, in Abating Obesity, Non-Alcoholic Steatohepatitis and Neurodegenerative diseases: A Narrative Review. Pharmaceutical Research. 2022; 39:1233-1248.
3. Merkus F W, Verhoef J C, N G Schipper, E Marttin. Nasal mucociliary clearance as a factor in nasal drug delivery. Adv. Drug Deliv Rev. 1998; 5; 29 (1-2): 13-38.
4. Pathak K. Mucoadhesion; A prerequisite or a constraint in nasal drug delivery? Int J Pharm Investig. 2011; 1 (2): 62-63.
5. Derakhshankhah H, Jafari S. Cell penetrating peptides: A concise review with emphasis on biomedical applications. Biomed Pharmacother. 2018; 108:1090-1096.

1. Use of a pharmaceutical composition for the manufacture of an intranasal drug for treatment or prevention of diabetes mellitus, obesity, nonalcoholic fatty liver disease (NAFLD), or neurodegenerative diseases, said pharmaceutical composition comprises an active ingredient and essential excipients, said active ingredient comprising 0.01% to 20% by weight (w/v) of semaglutide, or a pharmaceutically acceptable salt thereof, said pharmaceutical composition further comprising one or more permeation enhancers selected from a group consisting of cell penetrating peptides, tight junction modulating agents, and bioadhesive agents.

2. The use of claim 1, wherein said pharmaceutical composition is a liquid solution comprising an aqueous mixture of semaglutide, or a pharmaceutically acceptable salt, a permeation enhancer, a bioadhesive agent, a solubilising agent, a buffering agent, a chelator, a tonicity agent, and a preservative.

3. The use of claim 2, wherein the permeation enhancer is selected from a group consisting of penetratin, n-dodecylphosphocholine and dimethyl-β-cyclodextrin.

4. The use of claim 3, wherein the penetratin makes up 0.1% to 10% (w/v) of said composition.

5. The use of claim 3, wherein the n-dodecylphosphocholine makes up 0.1% to 10% (w/v) of said composition.

6. The use of claim 3, wherein the dimethyl-β-cyclodextrin makes up 0.1% to 50% (w/v) of said composition.

7. The use of claim 2, wherein the bioadhesive agent is selected from a group consisting of hydroxy propylmethyl cellulose, hydroxypropyl cellulose, carboxymethylcellulose sodium, chitosan, sodium hyaluronate, poloxamer 188/405, and carbopol 934P.

8. The use of claim 7, wherein the hydroxy propylmethyl cellulose makes up 0.05% to 5% (w/v) of said composition.

9. The use of claim 7, wherein the hydroxypropyl cellulose makes up 0.05% to 5% (w/v) of said composition.

10. The use of claim 7, wherein the carboxymethylcellulose sodium makes up 0.01% to 10% (w/v) of said composition.

11. The use of claim 7, wherein the chitosan makes up 0.01% to 5% (w/v) of said composition.

12. The use of claim 7, wherein the sodium hyaluronate makes up 0.01% to 5% (w/v) of said composition.

13. The use of claim 7, wherein the poloxamer 188 makes up 0.01% to 5% (w/v) of said composition, and poloxamer 405 makes up 0.1% to 30% (w/v) of said composition.

14. The use of claim 7, wherein the carbopol 934P makes up 0.05% to 10% (w/v) of said composition.

15. The use of claim 2, wherein the solubilising agent is selected from a group consisting of cyclodextrin or cyclodextrin derivatives.

16. The use of claim 15, wherein the cyclodextrin or cyclodextrin derivatives makes up 0.05% to 50% (w/v) of said composition.

17. The use of claim 2, wherein the buffering agent is selected from a group consisting of sodium di-hydrogen phosphate, di-sodium, phosphate, sodium citrate, and citric acid.

18. The use of claim 2, wherein the pharmaceutical composition has a pH in the range of 3.0 to 9.0.

19. The use of claim 2, wherein the chelator is EDTA-2Na.

20. The use of claim 19, wherein the EDTA makes up 0.1% to 5% (w/v) of said composition.

21. The use of claim 2, wherein the preservative is benzalkonium chloride.

22. The use of claim 21, wherein the benzalkonium chloride makes up 0.01% to 0.1% (w/v) of said composition.

23. The use of claim 2, wherein the tonicity agent is selected from a group consisting of sodium chloride, mannitol, and sorbitol.

24. The use of claim 2, wherein said liquid solution is formulated into nasal spray or nasal drop and for intranasal administration in mammals.

25. The use of claim 1, wherein said intranasal administration comprises a bottle and metered multi-dose pump.

26. The use of claim 2, wherein said pharmaceutical composition is formulated to intranasally deliver a volume of said composition of about 0.05 mL to 0.25 mL per spray.

27. The use of claim 2, wherein said intranasal administration is achieved by using a spray device intranasally delivering a dose of 0.005 mg to 50 mg semaglutide per spray.

28. The use of claim 1, wherein said pharmaceutical composition is formulated into a suspension, emulsion, bioadhesive or in-situ gel, microsphere, nanoparticle, or a self-emulsifying drug delivery system.

29. A method of treating or preventing diabetes mellitus, obesity, nonalcoholic fatty liver disease (NAFLD), or neurodegenerative diseases, said method comprising intranasal administration to a mammal subject in need therefor a therapeutically effective amount of the liquid solution manufactured by the use of claim 2.
